(19) United States
(12) Patent Application Publication
(10) Pub. No.: US 2008/0139608 A1
(43) Pub. Date: Jun. 12, 2008

(54) 2,6,8, Trisubstituted 1-deazapurines and their different uses

(75) Inventors: Lisa Chung Wai Chang, Newtown (AU); Johannes Brussee, Rijnsburg (NL); Adriaan Pieter IJzerman, Haarlem (NL)

Correspondence Address: TRASK BRITT, P.O. BOX 2550, SALT LAKE CITY, UT, 84110, US

(73) Assignee: Universiteit Leiden, Leiden (NL)

(21) Appl. No.: 11/634,806

(22) Filed: Dec. 6, 2006

(51) Int. Cl. A61K 31/4375; C07D 471/02

(52) U.S. Cl. 514/303; 546/118

(57) ABSTRACT

The invention provides a compound having the structure of general formula (I):

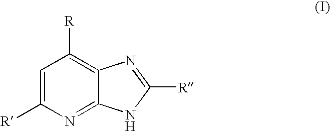

or a salt thereof,
wherein,
R represents hydrogen (except when R′=H), halogen, (substituted) alkyl except —CH₃, (substituted) alkenyl, (substituted) alkynyl, or (substituted) —(CH₂)ₙ-aryl;
R′ represents hydrogen (except when R=H), halogen, (substituted) alkyl except —CH₃, (substituted) alkenyl, (substituted) alkynyl, or (substituted) —CH₂)ₙ-aryl;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl; and
n is a number in the range from 0 to 10. The invention further provides pharmaceutical compositions comprising said compound, and the use of said compound to treat and/or prevent a variety of diseases.

### FIELD OF INVENTION

The present invention relates to a particular novel category of 2,6,8-trisubstituted 1-deazapurines, pharmaceutical compositions containing them and the use of the compounds and compositions.

### BACKGROUND OF THE INVENTION

The endogenous neuromodulator adenosine acts extracellularly via activation of specific membrane-bound receptors called P₁-purinoceptors. These adenosine receptors are divided into four subclasses: A₁, A_{2A}, A_{2B} and A₃ receptors. All four classes are coupled to the enzyme adenylate cyclase. Activation of the adenosine A₁ and A₃ receptors can lead to an inhibition of adenylate cyclase, while activated A_{2A} and A_{2B} receptors can stimulate adenylate cyclase. The adenosine receptors are ubiquitously distributed throughout the body and can modulate diverse physiological functions, including induction of sedation, relaxation of smooth muscle and vasodilation. Activation of these receptors by adenosine can, therefore, be of importance in many disease states. Accordingly, blocking these receptors can produce an effect leading to the prevention or treatment of many diseases. For example, the A_{2A} adenosine receptor antagonists are reported to have a beneficial effect on neurodegenerative diseases such as Parkinson's disease.¹ In recent years, a number of new and interesting ligands, which block the various adenosine receptor subtypes, have been synthesized. These ligands encompass bi- and tri-cyclic heteroaromatic systems, featuring 3-nitrogen tri-cyclic systems (e.g., the imidazoquinolines);² 4-nitrogen tri-cyclic systems (e.g., triazoloquinoxalines);³ 6-nitrogen tri-cyclic systems (e.g., the pyrazolotriazolopyrimidines);⁴ 2-nitrogen bi-cyclic systems (e.g., the naphthyridines);⁵ and 3-nitrogen bi-cyclic systems (e.g., deazaadenines).⁶

### SUMMARY OF THE INVENTION

It has now been found that a particular novel category of 2,6,8-trisubstituted 1-deazapurines can very attractively be used to treat adenosine receptor-mediated conditions.

1-Deazapurine is also known as 3H-imidazo[4,5-b]pyridine (and 4-azabenzimidazole), however, it is herein known as 1-deazapurine with the purine numbering system as given in FIG. 1.

Accordingly, the present invention relates to a compound of the general formula (I):

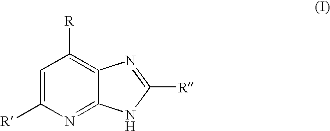

or a salt thereof,
wherein,
R represents hydrogen (except when R′=H), halogen, (substituted) alkyl except —CH₃, (substituted) alkenyl, (substituted) alkynyl, or (substituted) —(CH₂)ₙ-aryl;
R′ represents hydrogen (except when R=H), halogen, (substituted) alkyl except —CH₃, (substituted) alkenyl, (substituted) alkynyl, or (substituted) —CH₂)ₙ-aryl;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl; and
n is a number in the range from 0 to 10.

The compounds in accordance with the present invention block various adenosine receptor subtypes, thus establishing that diseases, such as, amongst others, cardiovascular, neurological, and immunological disorders, can very attractively be treated and/or prevented.

In the context of the present invention, the term “adenosine receptor-mediated conditions” is intended to include disease states or conditions characterized by their responsiveness to treatment with an adenosine receptor-mediating compound, e.g., a 2,6,8-trisubstituted 1-deazapurine derivative as described by general formula (I), where the treatment causes a significant diminishment of at least one symptom or effect of the state achieved with an adenosine receptor-mediating compound of the invention.

In the context of the present invention, by the term “alkyl” it is meant any saturated hydrocarbon, either branched or unbranched, comprising from 1 to about 30 carbon atoms. This includes straight-chained alkyl groups, branched-chained alkyl groups, cycloalkyl (alicyclic) groups, alkyl-substituted cycloalkyl groups, and cycloalkyl-substituted alkyl groups. This term further includes alkyl groups, which can further include oxygen, nitrogen, sulphur or phosphorous atoms replacing one or more carbons of the hydrocarbon backbone. In preferred embodiments, a straight or branched chain has 30 or less carbon atoms in its backbone and, more preferably, 20 carbon atoms or less. Likewise, preferred cycloalkyls have from three to ten carbons and, more preferably, three to seven carbons in the ring-structure.

In the context of the present invention, the term “—(CH₂)ₙ-hydroxyl” means a short straight alkyl chain between the hydroxyl group and the drawn structure, where n can range from zero up to and including ten.

In the context of the present invention, the terms “acyl,” “thio-acyl” and “seleno-acyl” refer to compounds of the kind “C(O)X,” “C(S)X,” and “C(Se)X,” respectively, where X in turn represents hydrogen, (substituted) alkyl, (substituted) alkenyl, (substituted) alkynyl, or (substituted) —(CH₂)ₙ-aryl.

In the context of the present invention, the term “—(CH₂)ₙ-aryl” means a short straight alkyl chain between the (substituted) aryl group and the drawn structure, where n can range from zero up to and including ten.

In the context of the present invention, the term “aryl” as used herein, refers to aromatic groups which can include five- and six-membered single-ring groups, with zero to four heteroatoms, for example, benzene, pyrrole, furan, thiophene, imidazole, triazole, tetrazole, pyrazole, pyridine, pyrazine, pyridazine and pyrimidine, and the like. The aryl groups can suitably include polycyclic fused aromatic groups, such as naphthyl, quinolyl, indolyl, benzoxazole, benzothiazole and the like. Those aryl groups containing heteroatoms may also be referred to as heteroaryls or heteroaromatics. The aromatic ring may be substituted at one or more ring positions, with such substituents as described herein. Aryl groups can also be fused or bridged with alicyclic or heteroalicyclic rings that are not aromatic.

In the context of the present invention, the term “substituted” is intended to include substituents replacing hydrogen on one or more of the carbons of a moiety. Such substituents suitably include, for example, halogen, hydroxyl, alkylcarbonyloxy, arylcarbonyloxy, alkoxycarbonyloxy, aryloxycarbonyloxy, carboxylate, alkylcarbonyl, alkyloxycarbonyl, aminocarbonyl, alkylthiocarbonyl, alkyoxyl, phosphate, phosphonate, phosphinato, cyano, amino (including alkylamino, dialkylamino, arylamino, diarylamino, alkylarylamino), acylamino, (including alkylcarbonylamino, arylcarbonylamino, carbamyl and ureido), amidino, imino, sulfhydryl, alkylthio, arylthio, thiocarboxylate, sulfates, sulfonato, sulfamoyl, sulfonamido, nitro, trifluoromethyl, azido, heterocyclyl, alkylaryl, or an aromatic or heteroaromatic moiety. It will be understood to those skilled in the art that the moieties substituted on the (unsaturated and saturated) carbon chain can themselves be substituted, if appropriate.

In the context of the present invention, the term “(substituted)” refers to the fact that a substitution in the chemical group is optional in nature. For example, “(substituted) alkyl” encompasses both substituted alkyls and unsubstituted alkyls; “(substituted) alkyl except —CH₃” encompasses both substituted alkyls except —CH₃ and unsubstituted alkyls except —CH₃; “(substituted) alkynyl” encompasses both substituted alkynyls and unsubstituted alkynyls; “(substituted) alkenyl” encompasses both substituted alkenyls and unsubstituted alkenyls; and “(substituted) —(CH₂)ₙ-aryl” encompasses both substituted —(CH₂)ₙ-aryls and unsubstituted —(CH₂)ₙ-aryls.

In the context of the present invention, the term “heteroatom” refers to an atom of any element other than carbon or hydrogen. Preferred heteroatoms are oxygen, nitrogen, sulphur and phosphorus.

In the context of the present invention, the terms “alkenyl” and “alkynyl” refer to unsaturated aliphatic groups analogous in length and possible substitution to the alkyls described above, but that contain at least one double or triple bond, respectively.

In the context of the present invention, the term “halogen” refers to an atom of group VII of the periodic table. Preferred halogens are fluorine, chlorine, bromine, and iodine.

In the context of the present invention, “salts” of the compound of the present invention are meant to include any physiologically acceptable salt. The term “physiologically acceptable salt” refers to any non-toxic alkali metal, alkaline earth metal, and ammonium salts commonly used in the pharmaceutical industry, including sodium, potassium, lithium, calcium, magnesium, barium ammonium and protamine zinc salts, which can be prepared by methods known in the art. The term also includes non-toxic “acid addition salts,” which are generally prepared by reacting the compounds of the present invention with a suitable organic or inorganic acid. The acid addition salts are those that retain the biological effectiveness and properties of the free bases and that are not biologically or otherwise undesirable. Examples include those derived from mineral acids and include, inter alia, hydrochloride, hydrobromic, sulphuric, nitric phosphoric, metaphosphoric and the like. Organic acids include, inter alia, tartaric, acetic, proprionic, citric, malic, malonic, lactic, fumaric, benzoic, cinnamic, mandelic, glycolic, gluconic, pyruvic, succinic, salicylic and arylsulfonic, e.g., p-toluenesulfonic, acids.

According to one embodiment of the invention, the substituent R represents hydrogen (except when R′=H), (substituted) alkyl except —CH₃, (substituted) alkenyl, (substituted) alkynyl, or (substituted) —(CH₂)ₙ-aryl; R′ represents hydrogen (except when R=H), (substituted) alkyl except —CH₃, (substituted) alkenyl, (substituted) alkynyl, or (substituted) —(CH₂)ₙ-aryl; R″ represents hydrogen, —(CH₂)ₙ-hydroxyl, acyl, (substituted) alkyl, (substituted) alkenyl, (substituted) alkynyl, or (substituted) —(CH₂)ₙ-aryl.

In a preferred embodiment, the present invention relates to a pharmaceutical composition comprising as active ingredient one or more compounds of the general formula (I):

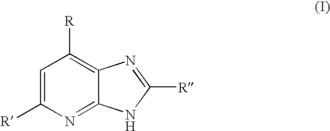

or a salt of the compound(s), wherein, R, R′, R″ have the meaning as defined hereinbefore.

### BRIEF DESCRIPTION OF THE DRAWING

FIG. 1: 1-deazapurine and the purine numbering system.

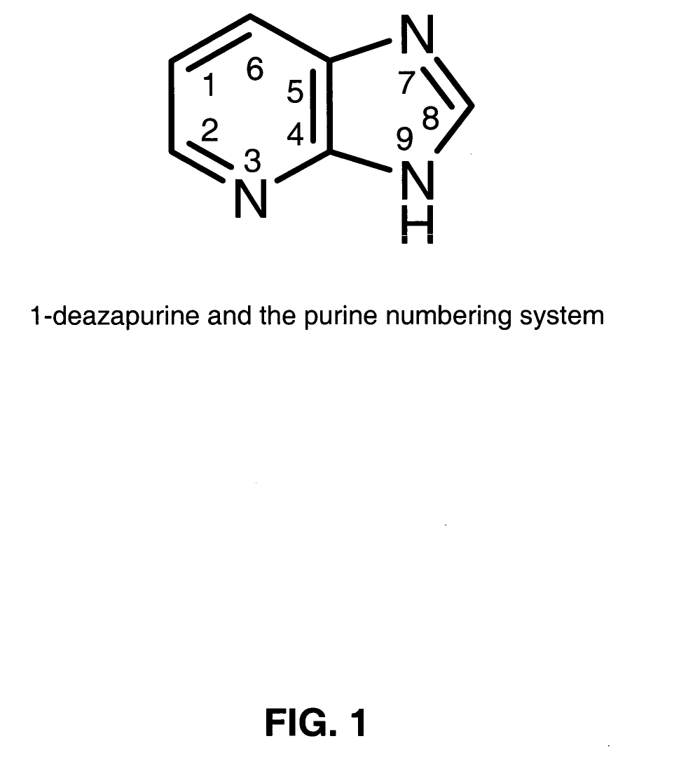

### DETAILED DESCRIPTION OF THE INVENTION

The present invention further relates to compounds of the general formula (I):

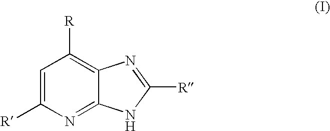

or a salt thereof,
wherein,
R represents hydrogen (except when R′=H), halogen, (substituted) alkyl except —CH₃, (substituted) alkenyl, (substituted) alkynyl, or (substituted) —(CH₂)ₙ-aryl;
R″ represents hydrogen (except when R=H), halogen, (substituted) alkyl except —CH₃, (substituted) alkenyl, (substituted) alkynyl, or (substituted) —(CH₂)ₙ-aryl;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl; and
n is a number in the range from 0 to 10.

More particularly, the present invention relates to compounds of general formula (I) or salts thereof,
R represents hydrogen (except when R′=H), halogen, alkyl except —CH₃, or (substituted) —(CH₂)ₙ-aryl;
R′ represents hydrogen (except when R′=H), halogen, alkyl except —CH₃, or (substituted) —(CH₂)ₙ-aryl;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl;
and n is a number in the range from 0 to 10.

According to another preferred embodiment of the present invention,
R represents a (substituted) —(CH₂)ₙ-aryl;
R′ represents a (substituted) —(CH₂)ₙ-aryl;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl; and
n is a number in the range from 0 to 10.

Preferably, R represents a (substituted) —(CH₂)ₙ-aryl;
R′ represents a (substituted) —(CH₂)ₙ-aryl;
R″ represents hydrogen, —(CH₂)ₙ-hydroxyl, halogen, (substituted) alkyl, (substituted) —(CH₂)ₙ-aryl; and
n is a number in the range from 0 to 10.

In another preferred embodiment,
R represents a (substituted) —(CH₂)ₙ-aryl;
R′ represents a halogen;
R″ represents hydrogen, —(CH₂)ₙ-hydroxyl, halogen, (substituted) alkyl, (substituted) —(CH₂)ₙ-aryl; and
n is a number in the range from 0 to 10.

In another preferred embodiment,
R represents a halogen;
R′ represents a (substituted) —(CH₂)ₙ-aryl;
R″ represents hydrogen, —(CH₂)ₙ-hydroxyl, halogen, (substituted) alkyl, (substituted) —(CH₂)ₙ-aryl; and
n is a number in the range from 0 to 10.

In a more preferred embodiment,
R represents a phenyl;
R′ represents a phenyl; R″ represents —(CH₂)ₙ-hydroxyl, (substituted) —(CH₂)ₙ-aryl, hydrogen or an alkyl; and
n is a number in the range from 0 to 10.

In another preferred embodiment,
R represents a halogen;
R′ represents a phenyl;
R″ represents —(CH₂)ₙ-hydroxyl, halogen, (substituted) —(CH₂)ₙ-aryl, hydrogen or an alkyl; and
n is a number in the range from 0 to 10.

In another preferred embodiment,
R represents a phenyl;
R′ represents a halogen;
R″ represents —(CH₂)ₙ-hydroxyl, halogen, (substituted) —(CH₂)ₙ-aryl, hydrogen or an alkyl; and
n is a number in the range from 0 to 10.

In yet another preferred embodiment,
R represents a (substituted) alkyl except —CH₃;
R′ represents a (substituted) alkyl except —CH₃;
R″ represents hydrogen, halogen, —(CH₂)ₙ-hydroxyl, (substituted) —(CH₂)ₙ-aryl, (substituted) alkyl; and
n is a number in the range from 0 to 10.

In another preferred embodiment,
R represents a (substituted) alkyl except —CH₃;
R′ represents a (substituted) alkyl except —CH₃;
R″ represents hydrogen, halogen, —(CH₂)ₙ-hydroxyl, (substituted) —(CH₂)ₙ-aryl, (substituted) alkyl; and
n is a number in the range from 0 to 10.

In another preferred embodiment,
R represents halogen;
R′ represents a (substituted) alkyl except —CH₃;
R″ represents hydrogen, —(CH₂)ₙ-hydroxyl, halogen, (substituted) alkyl; and
n is a number in the range from 0 to 10.

In another preferred embodiment,
R represents a (substituted) alkyl except —CH₃;
R′ represents a halogen, —(CH₂)ₙ-hydroxyl, halogen, (substituted) alkyl, (substituted) —(CH₂)ₙ-aryl; and
n is a number in the range from 0 to 10.

Preferably, the compound according to the present invention is chosen from the group consisting of 2,6-Diphenyl-1-deazapurine, 2,6-Bis(4-chlorophenyl)-1-deazapurine, 2,6-Bis(4-tolyl)-1-deazapurine, 2,6-Bis(4-methoxyphenyl)-1-deazapurine, 2-(4-chlorophenyl)-6-phenyl-1-deazapurine, 2-Tolyl-6-phenyl-1-deazapurine, 2-(4-methoxyphenyl)-6-phenyl-1-deazapurine, 6-Chloro-2,8-diphenyl-1-deazapurine, 2-Phenyl-6-chloro-8-propyl-1-deazapurine, 2-Phenyl-6-chloro-8-isopropyl-1-deazapurine, 2-Phenyl-6-chloro-8-cyclopentyl-1-deazapurine, 2-Phenyl-6-chloro-8-cyclohexyl-1-deazapurine, 2,6,8-Triphenyl-1-deazapurine, 8-Propyl-2,6-diphenyl-1-deazapurine, 8-Isopropyl-2,6-diphenyl-1-deazapurine, 8-Cyclopentyl-2,6-diphenyl-1-deazapurine, 8-Cyclohexyl-2,6-diphenyl-1-deazapurine, 8-Cyclohexyl-6-(4-chlorophenyl)-2-phenyl-1-deazapurine, 8-Cyclohexyl-6-(3,4-dichlorophenyl)-2-phenyl-1-deazapurine, 8-Cyclohexyl-6-(4-tolyl)-2-phenyl-1-deazapurine, 8-Cyclohexyl-6-(4-methoxyphenyl)-2-phenyl-1-deazapurine, 8-Furan-2-yl-2,6-diphenyl-1-deazapurine, 2,6-Diphenyl-8-thien-2-yl-1-deazapurine or a salt thereof.

More preferably, the compound according to the present invention is chosen from the group consisting of 2,6-Diphenyl-1-deazapurine, 2,6,8-Triphenyl-1-deazapurine, 8-Propyl-2,6-diphenyl-1-deazapurine, 8-Isopropyl-2,6-diphenyl-1-deazapurine, 8-Cyclopentyl-2,6-diphenyl-1-deazapurine, 8-Cyclohexyl-2,6-diphenyl-1-deazapurine, 8-Cyclohexyl-6-(4-chlorophenyl)-2-phenyl-1-deazapurine, 8-Cyclohexyl-6-(3,4-dichlorophenyl)-2-phenyl-1-deazapurine, 8-Cyclohexyl-6-(4-tolyl)-2-phenyl-1-deazapurine, 8-Cyclohexyl-6-(4-methoxyphenyl)-2-phenyl-1-deazapurine or a salt thereof.

Most preferably, the compound comprises 8-Cyclopentyl-2,6-diphenyl-1-deazapurine, 8-Isopropyl-2,6-diphenyl-1-deazapurine, 8-Cyclohexyl-2,6-diphenyl-1-deazapurine, 8-Furan-2-yl-2,6-diphenyl-1-deazapurine, 2,6-Diphenyl-8-thien-2-yl-1-deazapurine 8-propyl-2,6-diphenyl-1-deazapurine, 8-methyl-2,6-diphenyl-1-deazapurine or a salt thereof.

The compounds of the present invention may be prepared by several synthetic procedures. For example, the synthetic route to obtain some 2,6,8-trisubstituted derivatives is depicted in the scheme hereinbelow:

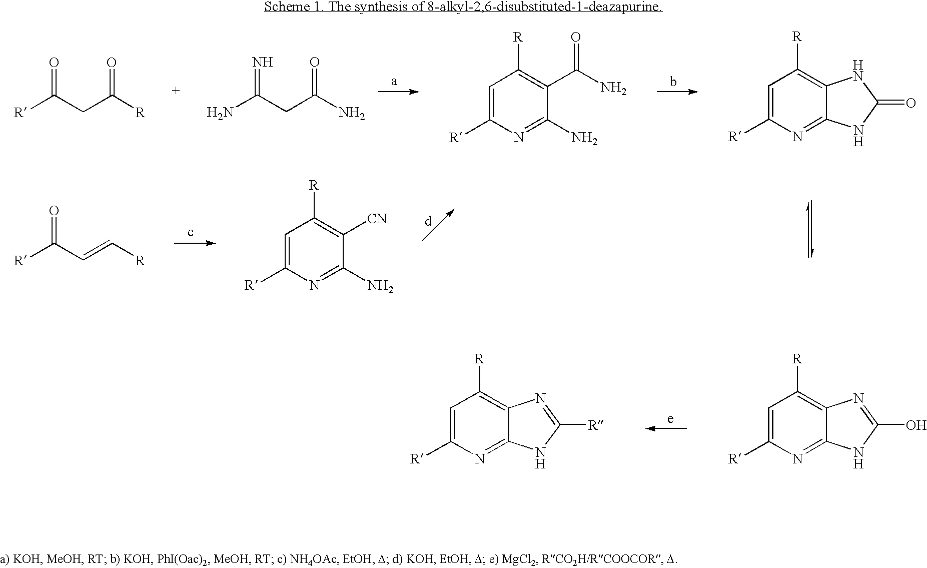

According to this scheme, the synthesis began with the reaction of the commercially available dibenzoylmethane with malonamamidine in a similar fashion to a route described by Senanayake et al.⁷ This was a low-yielding step. We thus sought the construction of the nicotinamide in a different manner. A two-step approach employing a chalcone and malononitrile in the presence of ammonium acetate formed a cyanopyridine, which upon hydrolysis, gave the target nicotinamide (Scheme 6.9).⁸ This route also allows regioselective substitution about the pyridine ring if so desired, a feature not possible in the original route with the diketone. The Hoffman rearrangement proceeded without any significant problems and a substantial amount of the 2,6-diphenyl-8-hydroxy-1-deazapurine (3) could be made. Substitution of the 8-OH was performed as described by Senanayake et al.⁷ using a mixture of an acid and the corresponding anhydride in the presence of MgCl₂. A main difference introduced was the use of the microwave for this procedure. Aromatic substituents at the 2- and 6-positions of the deazapurine ring significantly lower its reactivity, and the employment of conventional heating methods resulted in very low yields and considerable quantities of by-products, which made the final product difficult to isolate. Using the microwave, rapid heating of the sealed vessel quickly created very high temperatures and elevated pressures. This method improved the synthesis, dramatically leading to better yields and easier isolation of the final target products.

Accordingly, the present invention also relates to a process for preparing a compound according to present invention, which process comprises the steps of:
(a) i) reacting a compound having the structure R′COCH₂COR with malonamamidine, or a salt thereof or, alternatively,
   ii) reacting a compound having the structure R′COCH═CHR with malononitrile, or a salt thereof, yielding intermediate structure:

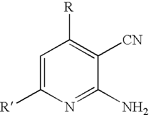

with subsequent hydrolysis of the cyano group,
to finally form a product having the structure:

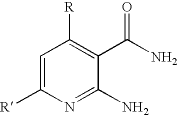

wherein,
R represents hydrogen (except when R′=H), halogen, (substituted) alkyl except —CH₃, (substituted) alkenyl, (substituted) alkynyl, or (substituted) —(CH₂)ₙ-aryl; and
R′ represents hydrogen (except when R=H), halogen, (substituted) alkyl except —CH₃, (substituted) alkenyl, (substituted) alkynyl, or (substituted)-CH₂)ₙ-aryl; and
n is a number in the range from 0 to 10;
(b) subjecting the final product formed in step (a) to a Hoffmann rearrangement reaction to form a product having the structure:

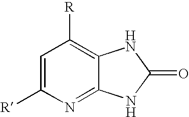

isomeric to structure:

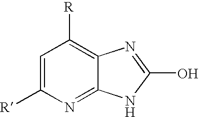

wherein, R, R′ and n have the meaning as defined in step (a);
(c) subjecting the product formed in step (b) to a mixture of an appropriate acid and its corresponding acid anhydride to form a product having the structure:

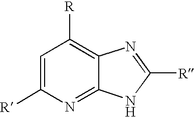

wherein,
R R′ and n have the meaning as defined in step (a), and
R represents hydrogen (except when R′=H), halogen, (substituted) alkyl except —CH₃, (substituted) alkenyl, (substituted) alkynyl, or (substituted) —(CH₂)ₙ-aryl; and
R′ represents hydrogen (except when R=H), halogen, (substituted) alkyl except —CH₃, (substituted) alkenyl, (substituted) alkynyl, or (substituted) —CH₂)ₙ-aryl; and
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl.

The present invention also relates to a pharmaceutical composition comprising as active ingredient one or more compounds according to the present invention. The compound according to the present invention can be used as such. However, also a salt or a solvate of the compound may be used. It will be understood that such salt or solvate should be pharmaceutically acceptable. The skilled person will further understand that the pharmaceutical composition will also comprise a suitable pharmaceutical carrier.

The present invention further relates to the use of a compound according to the present invention for treating and/or preventing a disorder in which the adenosine receptors are involved.

The present invention also relates to the use of a compound according to the present invention for the manufacture of a medicament for the treatment and/or prevention of a disorder in which the adenosine receptors are involved.

In addition, the present invention relates to a method for treating and/or preventing a disorder in which the interaction with the adenosine receptors is beneficial, which method comprises administering to a subject in need of such treatment an effective dose of a pharmaceutical composition in accordance with the present invention.

Suitably, the disorder can be chosen from the group of diseases consisting of, amongst others, cardiovascular, neurological, immunological disorders, cancers and infection conditions. The compounds according to the present invention are particularly effective for treating and/or preventing kidney, heart and central nervous system (CNS) afflictions.

As will be detailed in Table 2, the compounds of the present invention are biologically active.

The term “biologically active” indicates that the compound of the present invention has some sort of a biological activity, for example, a measurable effect on a target receptor. As will be detailed hereinafter, the compound of the present invention may block the biological action of adenosine receptors, thus acting as adenosine receptor antagonists.

The term “antagonist” as used herein refers to a molecule that binds to a receptor without activating the receptor. It competes with the endogenous ligand for this binding site and, thus, reduces the ability of the endogenous ligand to stimulate the receptor.

Thus, the present invention also relates to pharmaceutical compositions comprising as active ingredient one or more of a compound of the general formula (I):

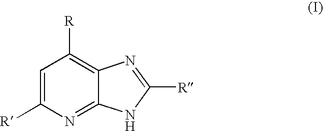

or a salt thereof,
wherein,
R represents hydrogen (except when R′=H), halogen, (substituted) alkyl except —CH₃, (substituted) alkenyl, (substituted) alkynyl, or (substituted) —(CH₂)ₙ-aryl;
R′ represents hydrogen (except when R=H), halogen, (substituted) alkyl except —CH₃, (substituted) alkenyl, (substituted) alkynyl, or (substituted) —(CH₂)ₙ-aryl;
R″ represents hydrogen, —(CH₂)ₙ-hydroxyl, halogen, acyl, thio-acyl, seleno-acyl, (substituted) alkyl, (substituted) alkenyl, (substituted) alkynyl, or (substituted) —(CH₂)ₙ-aryl; and
n is a number in the range from 0 to 10.

In the pharmaceutical composition according to the present invention, the active ingredient is present in an effective amount. The term “effective amount” for the purposes described herein is that determined by such considerations as are known to those versed in the art. The amount must be sufficient to achieve a desired therapeutic effect, e.g., to treat a disease or disorder.

The terms “treat,” “treating” and “treatment” refer to the administering of a therapeutic amount of the compound or pharmaceutical composition of the present invention that is effective to ameliorate undesired symptoms associated with a disease, to prevent the manifestation of such symptoms before they occur, to slow down the progression of a disease, to slow down the deterioration of symptoms, to slow down the irreversible damage caused by the chronic stage of a disease, to lessen the severity of, or cure, a disease, to improve survival rate or more rapid recovery, to prevent the disease from occurring, or a combination of two or more of the above.

The terms “modulate,” “modulating,” and “modulation” are intended to include preventing, eradicating, or inhibiting the resulting increase of undesired physiological activity associated with the stimulation of an adenosine receptor, e.g., in the context of the therapeutic methods of this invention. In another embodiment, the term “modulate” includes antagonistic effects, e.g., diminishment of the activity or production of mediators that result from the (over)-stimulation of adenosine receptor(s).

The disease is preferably associated with the biological action of one or more adenosine receptors wherein the compound of the present invention acts as an adenosine receptor antagonist. For example, antagonists of A₁ receptors have been implicated as compounds that may be used in the treatment of cardiac, renal and sleep disorders.

The pharmaceutical composition of the present invention may further comprise pharmaceutically acceptable additives.

Further, the term “pharmaceutically acceptable additives” as used herein refers to any substance combined with the compound and include, without being limited thereto, diluents, excipients, carriers, solid or liquid fillers or encapsulating materials that are typically added to formulations to give them a form or consistency when it is given in a specific form, e.g., in tablet form, as a simple syrup, aromatic powder, and other various elixirs. The additives may also be substances for providing the formulation with stability, sterility and isotonicity (e.g., antimicrobial preservatives, antioxidants, chelating agents and buffers), for preventing the action of microorganisms (e.g., antimicrobial and antifungal agents, such as parabens, chlorobutanol, phenol, sorbic acid and the like), or for providing the formulation with an edible flavor, etc.

Preferably, the additives are inert, non-toxic materials that do not react with the active ingredient of the invention. Yet, the additives may be designed to enhance the binding of the agent to its receptor. Further, the term “additive” may also include adjuvants, which, by definition, are substances affecting the action of the active ingredient in a predictable way. The additive can be any of those conventionally used and are only limited by chemico-physical considerations, such as solubility and lack of reactivity with the compound of the invention, and by route of administration. The active agent of the invention may be administered orally to the patient. Conventional methods, such as administering the compound/s in tablets, suspensions, emulsions, capsules, powders, syrups and the like, are usable. For oral administration, the composition of the invention may contain additives for facilitating oral delivery of the compound/s of the invention. Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin-type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, and pharmacologically compatible carriers. Lozenge forms can comprise the active agent in a flavor, usually sucrose and acacia or tragacanth, as well as pastilles comprising the active ingredient in an inert base, such as gelatin and glycerine, or sucrose and acacia, emulsions, gels, and the like. Such additives are as such known in the art.

Alternatively, the compound/s may be administered to the patient parenterally. In this case, the composition will generally be formulated in a unit dosage injectable form (solution, suspension, emulsion). Pharmaceutical formulation suitable for injection may include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, lipid polyethylene glycol and the like), suitable mixtures thereof and vegetable oils.

Proper fluidity can be maintained, for example, by the use of a coating such as lecithin, by the maintenance of the required particle size in the case of dispersion and by the use of surfactants. Non-aqueous vehicles, such as cottonseed oil, sesame oil, olive oil, soybean oil, corn oil, sunflower oil, or peanut oil and ester, such as isopropyl myristate, may also be used as solvent systems for the composition of the present invention.

Suitable fatty acids for the use in parenteral formulations include oleic acid, stearic acid, and isostearic acid. Ethyl oleate and isopropyl myristate are examples of suitable fatty acid esters.

Suitable detergents for use in parenteral formulations include fatty alkali metal, ammonium, and triethanolamine salts, and suitable detergents include (a) cationic detergents, such as, for example, dimethyl dialkyl ammonium halides, and alkyl pyridinium halides, (b) anionic detergents, such as, for example, alkyl, aryl, and olefinic sulfonates, alkyl, olefin, ether and monoglyceride sulfates, and sulfosuccinates, (c) non-ionic detergents, such as, for example, fatty amine oxides, fatty acid alkanolamides, and polyoxy-ethylenepolypropylene copolymers, (d) amphoteric detergents, such as, for example, alkyl-β-aminopropionates, and 2-alkyl-imidazoline quarternary ammonium salts, and mixtures thereof.

Further, in order to minimize or eliminate irritation at the site of injection, the compositions may contain one or more non-ionic surfactants having a hydrophile-lipophile balance (HLB) from about 12 to about 17. Suitable surfactants include polyethylenesorbitan fatty acid esters, such as sorbitan monooleate and the high molecular weight adducts of ethylene oxide with a hydrophobic base, formed by the condensation of propylene oxide with propylene glycol.

The choice of an additive will be determined in part by the particular compound of the present invention, as well as by the particular method used to administer the composition.

Notwithstanding the above, the composition of the present invention may include one or more of the compounds of the present invention and may compromise other biologically active substances, to provide a combined therapeutic effect.

The compounds and compositions of the present invention as set forth hereinabove and below are administered and dosed in accordance with good medical practice, taking into account the clinical conditions of the individual patient, the site and method of administration, scheduling of administration, individual's age, sex, body weight and other factors known to medical practitioners.

The dose may be single doses or multiple doses over a period of several days. The treatment generally has a length proportional to the length of the disease process and drug effectiveness and the individual species being treated. Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments, until the optimum effect under the circumstances is reached. Exemplary dosages range from about 0.01 mg/kg body weight to about 10 mg/kg body weight of the subject being treated per day.

The invention has been described in an illustrative manner, and it is to be understood that the terminology that has been used is intended to be in the nature of words of description rather than of limitation. Obviously, many modifications and variations of the present invention are possible in light of the above teaching. It is, therefore, to be understood that within the scope of the appended claims, the invention may be practiced otherwise than as specifically described hereinafter.

Throughout this application, various publications are referred to by a number. Full citations for the publications are listed hereinafter. The disclosure of these publications in their entireties is hereby incorporated by reference into the application in order to more fully describe the state of the art to which this invention pertains.

### SPECIFIC EXAMPLES

### 2,6-Diphenyl-8-substituted-1-deazapurines

This invention is further described in the following specific examples, which do not limit the scope of the invention described in the claims.

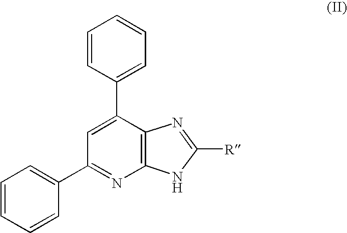

The examples detailed here of the general formula (II) are synthesized according to the route detailed below in Scheme 3.

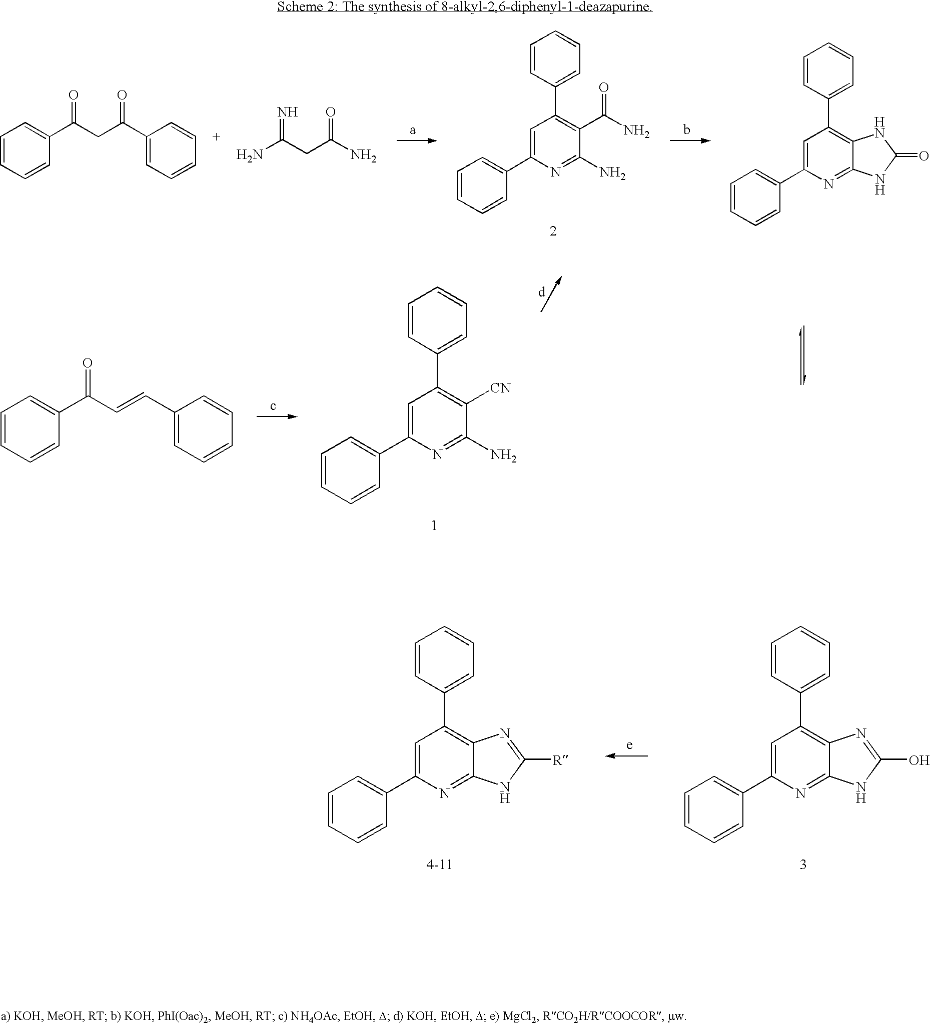

### Chemistry—General

Chemicals and Solvents. All reagents were obtained from commercial sources and all solvents were of an analytical grade.

Chromatography. Thin-layer chromatography (TLC) was carried out using Merck silica gel plastic backed F₂₅₄ plates, visualized under UV (254 nm).

Instruments and Analysis. Elemental analyses were performed for C,H,N (Leiden Institute of Chemistry, Leiden University, The Netherlands). ¹H and ¹³C NMR spectra were recorded on a Bruker AC 200 (¹H NMR, 200 MHz; ¹³C NMR, 50.29 MHz) spectrometer with tetramethylsilane (TMS) as an internal standard. Chemical shifts are reported in ppm (6) relative to this. Melting points were determined on a Büchi melting point apparatus and are uncorrected. Mass Spectra were measured on a Finnigan MAT TSQ-70 spectrometer equipped with an electrospray interface for ESI experiments. Spectra were collected by constant infusion of the analyte dissolved in methanol. ESI is a soft ionization technique resulting in protonated, sodiated species in positive ionization mode and deprotonated species in the negative ionization mode.

### Synthetic Procedures

### 2-Amino-4,6-diphenyl-nicotinonitrile (1)⁸

Chalcone (benzylideneacetophenone) (20.8 g, 100 mmol), malononitrile (6.6 g, 100 mmol, 1 eq.), ammonium acetate (61.6 g, 800 mmol, 8 eq.) were dissolved in EtOH (15 mL) and refluxed for five hours, whereupon, no starting material was evident by TLC. The reaction mixture was allowed to cool to room temperature and the solvents evaporated to leave a yellow solid. This was taken up in approximately 10 mL of hot EtOH and filtered. The remaining off-white solids were then washed with petroleum ether. This was recrystallized from hot ethanol to give white crystals. Yield 40% mp 170-174° C.; ¹H NMR δ(DMSO): 8.17-8.14 (m, 2H, Ph), 7.73-7.50 (m, 8H, Ph), 7.30 (s, 1H, py-H), 7.05 (br s, 2H, NH₂). ¹³C-NMR δ(DMSO): 160.8, 158.6, 154.9, 137.5, 137.0, 130.1, 129.5, 128.6, 128.3, 127.2, 117.0, 109.2. MS (ES⁺): 272.0 Da. Anal. (C₁₈H₁₃N₃.0.16Hex) C, H, N.

### 2-Amino-4,6-diphenyl-nicotinamide (2)⁹

2-Amino-diphenyl-nicotinonitrile (10.8 g, 39.7 mmol) was refluxed in 20% KOH_{(aq.)} (30 g) and EtOH (150 mL) for 22 hours. H₂O (100 mL) was then added and the reaction mixture allowed to stand, upon which crystallization occurred. This yellow solid was collected and dried in vacuo at 40° C. Quantitative yield. ¹H NMR δ(DMSO): 8.03-8.00 (m, 2H, Ph), 7.62-7.58 (m, 2H, Ph), 7.47-7.31 (m, 6H, Ph), 6.98 (s, 1H, py-H).

### 2,6-Diphenyl-8-hydroxy-1-deazapurine (3)¹⁰

2-Amino-4,6-diphenyl-nicotinamide (12.75 g, 44 mmol) was dissolved in a solution of KOH (6.43 g, 110 mmol, 2.5 eq.) in MeOH (300 mL) and stirred for 30 minutes at room temperature. The reaction mixture was then cooled to −5° C., iodobenzenediacetate (14.2 g, 44 mmol, 1 eq.) added, allowed to warm to room temperature and left to stand for 40 hours. The reaction mixture was then further dissolved/diluted with methanol (175 mL) and H₂O (100 mL) and the solution neutralized with 1M HCl, then stirred with cyclohexane to remove traces of the iodobenzenediacetate. The hexane layer was then separated and the remaining MeOH/H₂O layer concentrated to leave a yellow solid. Recrystallized from EtOH to give a white solid. Yield 27%. mp 240-244° C.; ¹H NMR δ(DMSO): 8.02-7.98 (m, 2H, Ph), 7.71-7.67 (m, 2H, Ph), 7.60-7.36 (m, 7H, C1-H+Ph). ¹³C-NMR δ(DMSO): 155.2, 147.9, 145.8, 139.1, 135.2, 129.0, 128.6, 128.3, 128.0, 126.2, 120.3. MS (ES⁺): 287.6 Da. Anal. (C₁₈H₁₃N₃O.0.4EtOH)C, H, N.

### General procedure for the preparation of 8-alkyl-2,6-diphenyl-1-deazapurines (4-11)

2,6-Diphenyl-8-hydroxy-1-deazapurine (3), (200 mg, 0.7 mmol), carboxylic acid (1.5 mL, 16.2 mmol, 23 eq.), carboxylic acid anhydride (1.5 mL, 9.0 mmol, 13 eq.) and MgCl₂ (66 mg, 0.7 mmol, 1 eq.) were heated in a sealed vessel at 180° C. for ten hours. The reaction mixture was then concentrated, co-distilling with water to remove the excess acid/anhydride. Co-distilling with toluene removed the last traces of water. The crude material was then purified by column chromatography on SiO₂, eluting with CH₂Cl₂ and MeOH (99:1), then recrystallized.

### 2,6-Diphenyl-8-methyl-1-deazapurine (4)

Yield 87%. mp 188-192° C.; ¹H NMR δ(CDCl₃): 8.16-8.03 (m, 4H, Ph), 7.77 (s, 1H C1-H), 7.59-7.47 (m, 6H, Ph), 2.01 (s, 3H, CH₃). ¹³C-NMR δ(CDCl₃): 153.9, 151.8, 150.5, 140.3, 139.6, 136.3, 133.1, 129.0, 128.8, 128.6, 127.7, 114.6, 14.9. MS (ES⁺): 286.0 Da. Anal. (C₁₉H₁₅N₃. 0.02CHCl₃) C, H, N.

### 2,6-Diphenyl-8-ethyl-1-deazapurine (5)

Yield 36%. mp 188-192° C.; ¹H NMR δ(CDCl₃+MeOD): 7.93-7.87 (m, 4H, Ph), 7.62 (s, 1H C1-H), 7.42-7.36 (m, 6H, Ph), 2.72 (q, 2H, J=7.3 Hz, CH₂), 1.22 (t, 3H, J=7.3 Hz, CH₃). ¹³C-NMR δ(CDCl₃+MeOD): 159.1, 152.1, 139.8, 136.0, 130.8, 128.7, 128.6, 128.5, 128.3, 127.1, 114.7, 22.5, 12.0. MS (ES⁺): 300.0 Da. Anal. (C₂₀H₁₇N₃.0.4H₂O)C, H, N.

### 2,6-Diphenyl-8-propyl-1-deazapurine (6)

Yield 29%. mp 156-158° C.; ¹H NMR δ(MeOD): 8.05-8.04 (m, 2H, Ph), 8.00-7.89 (m, 2H, Ph), 7.70 (s, 1H C₁-H), 7.54-7.35 (m, 6H, Ph), 2.88 (t, 2H, J=7.3 Hz, CH₂), 1.88 (m, 2H, CH₂), 1.01 (t, 3H, J=7.3 Hz, CH₃). ¹³C-NMR δ(CDCl₃): 153.4, 151.6, 150.0, 141.3, 137.4, 129.7, 129.4, 128.0, 115.3, 31.8, 22.6, 13.8. MS (ES⁺): 314.0 Da. Anal. (C₂₁H₁₉N₃. 0.3EtOH)C, H, N.

### 2,6-Diphenyl-8-isopropyl-1-deazapurine (7)

Yield 71%. mp 232-234° C.; ¹H NMR δ(MeOD+CDCl₃): 8.01-7.99 (m, 2H, Ph), 7.97-7.87 (m, 2H, Ph), 7.66 (s, 1H C₁-H), 7.49-7.37 (m, 6H, Ph), 3.29-3.22 (m, 1H, CH), 1.43 (d, 6H, J=7.3 Hz, 2×CH₃). ¹³C-NMR δ(DMSO): 162.1, 150.4, 150.2, 139.6, 137.1, 136.1, 131.6, 129.1, 128.7, 128.5, 128.3, 126.7, 112.2, 29.0, 21.1. MS (ES⁺): 314.0 Da. Anal. (C₂₁H₁₉N₃. 0.1 CHCl₃) C, H, N.

### 2,6-Diphenyl-8-isobutyl-1-deazapurine (8)

Yield 65%. mp 179-181° C.; ¹H NMR δ(CDCl₃): 8.22-8.19 (m, 2H, Ph), 8.10-8.06 (m, 2H, Ph), 7.80 (s, 1H C1-H), 7.55-7.44 (m, 6H, Ph), 2.24 (d, 2H, J=7.3 Hz, CH₂), 1.86-1.80 (m, 1H, CH), 0.64 (d, 6H, J=5.8 Hz, 2×CH₃). ¹³C-NMR δ(CDCl₃): 157.2, 151.7, 150.4, 140.4, 139.5, 136.3, 133.1, 129.2, 128.7, 17.7, 114.6, 38.1, 22.1. MS (ES⁺): 328.0 Da. Anal. (C₂₂H₂₁N₃. 0.6H₂O)C, H, N.

### 2,6-Diphenyl-8-isopentyl-1-deazapurine (9)

Yield 39%. mp 196-197° C.; ¹H NMR δ(CDCl₃): 8.27-8.24 (m, 2H, Ph), 8.11-8.07 (m, 2H, Ph), 7.82 (s, 1H C₁-H), 7.57-7.43 (m, 6H, Ph), 2.37-2.30 (m, 1H, CH), 1.53-1.43 (m, 4H, 2×CH₂), 0.70-0.63 (m, 6H, 2×CH₃). ¹³C-NMR δ(CDCl₃): 160.3, 151.8, 150.0, 140.4, 139.2, 136.4, 132.7, 129.3, 128.9, 128.8, 128.5, 127.5, 114.3, 43.5, 26.6, 11.7. MS (ES⁺): 342.0 Da. Anal. (C₂₃H₂₃N₃. 0.05 CHCl₃) C, H, N.

### 2,6-Diphenyl-8-tButyl-1-deazapurine (10)

Yield 76%. mp 233-234° C.; ¹H NMR δ(CDCl₃): 8.33-8.29 (m, 2H, Ph), 8.01-7.98 (m, 2H, Ph), 7.82 (s, 1H C1-H), 7.55-7.41 (m, 6H, Ph), 1.44 (s, 9H, 3×CH₃). ¹³C-NMR δ(CDCl₃): 163.8, 152.1, 150.1, 140.2, 139.0, 138.2, 136.2, 132.3, 129.3, 128.9, 128.8, 128.7, 128.4, 128.3, 127.4, 114.1, 33.6, 28.9. MS (ES⁺): 328.0 Da. Anal. (C₂₂H₂₁N₃. 0.04 CHCl₃) C, H, N.

### 8-Cyclopentyl-2,6-diphenyl-1-deazapurine (11)

Yield 32%. mp 236° C.; ¹H NMR δ(MeOD+CDCl₃): 8.03-7.96 (m, 3H, Ph+NH), 7.89 (s, 1H, C1-H), 7.71-7.64 (m, 2H, Ph), 7.57-7.40 (m, 6H, Ph), 3.41-3.35 (m, 1H, CH), 2.21-2.19 (m, 2H, CH₂), 1.99-1.95 (m, 2H, CH₂), 1.94-1.89 (m, 2H, CH₂), 1.77-1.76 (m, 2H, CH₂). ¹³C-NMR δ(MeOD+CDCl₃): 157.1, 144.7, 142.6, 141.1, 133.6, 133.4, 133.2, 132.2, 131.9, 131.7, 131.3, 119.6, 44.9, 37.1, 30.3. MS (ES⁺): 340.3 Da. Anal. (C₂₃H₂₁N₃. 1.2H₂O)C, H, N.

### 2,6-Diphenyl-8-cyclohexyl-1-deazapurine (12)

Yield 49%. mp 210-213° C.; ¹H NMR δ(CDCl₃): 8.32-8.22 (m, 2H, Ph), 8.11-8.08 (m, 2H, Ph), 7.83 (s, 1H C1-H), 7.55-7.45 (m, 6H, Ph), 2.51-2.44 (m, 1H, CH), 1.10-1.06 (m, 10H, cHex). ¹³C-NMR δ(CDCl₃): 161.4, 151.8, 150.2, 140.2, 139.4, 136.4, 129.3, 129.0, 128.8, 128.6, 127.5, 114.3, 38.6, 31.3, 25.7. MS (ES⁺): 354.0 Da. Anal. (C₂₄H₂₃N₃. 0.5 EtOH)C, H, N.

TABLE 1
Elemental Analysis

| | | Elemental Analysis | | |
|---|---|---|---|---|
| Compound No. | Molecular formula | C % | H % | N % |
| 3 | C₁₈H₁₃N₃O•0.4EtOH Calc. | 73.97 | 5.03 | 13.82 |
| | Found | 73.95 | 4.64 | 13.60 |
| 4 | C₁₉H₁₅N₃•0.02CHCl₃ | 79.32 | 5.26 | 14.59 |
| | | 79.30 | 5.49 | 14.63 |
| 5 | C₂₀H₁₇N₃•0.4H₂O | 78.14 | 5.87 | 13.67 |
| | | 78.08 | 6.03 | 14.07 |
| 6 | C₂₁H₁₉N₃•0.3EtOH | 79.19 | 6.43 | 12.80 |
| | | 79.33 | 6.49 | 12.52 |
| 7 | C₂₁H₁₉N₃•0.1CHCl₃ | 78.01 | 5.93 | 12.94 |
| | | 77.99 | 6.13 | 12.99 |
| 8 | C₂₂H₂₁N₃•0.6H₂O | 78.27 | 6.61 | 12.95 |
| | | 78.21 | 6.47 | 12.74 |
| 9 | C₂₃H₂₃N₃•0.05CHCl₃ | 79.73 | 6.69 | 12.10 |
| | | 79.70 | 6.98 | 12.10 |
| 10 | C₂₂H₂₁N₃•0.04CHCl₃ | 79.74 | 6.39 | 12.66 |
| | | 79.73 | 6.77 | 12.57 |
| 11 | C₂₃H₂₁N₃•1.2H₂O | 76.36 | 6.54 | 11.62 |
| | | 76.31 | 6.37 | 11.88 |
| 12 | C₂₄H₂₃N₃•0.5 EtOH | 79.88 | 6.93 | 11.21 |
| | | 79.82 | 7.17 | 11.47 |

### Biology

A primary function of certain cell surface receptors is to recognize appropriate ligands. Accordingly, we performed radioligand binding studies to establish the degree to which the compound binds to the receptor.

Radioligand Binding Studies [³H]DPCPX was purchased from Amersham. All compounds made were tested in radioligand binding assays to determine their affinities at the human adenosine A₁ receptor. The affinities at the A₁ receptors were determined on CHO cells expressing the human receptors, using [³H]DPCPX as the radioligand according to a previously described method.¹¹

Data Analysis Competition binding data were fit to a single-site binding model and plotted using the software package Prism (Graph Pad, San Diego, Calif., USA). The Cheng-Prusoff equation Kᵢ=IC₅₀/(1+[I]/K_{d}) was used to calculate Kᵢ values, where Kᵢ is the affinity constant for the competing ligand, [I] is the concentration of the free radioligand, and K_{d} is the affinity constant for the radioligand.

### Structure Activity Relationships

In Table 2, results of the radioligand binding assays at the A₁ receptor are displayed, the substituents are defined hereinabove and below with reference to the compound of general formula (II). This 1-deazapurine core with the 2,6,8-trisubstitution pattern has surprising efficacy at the adenosine A₁ receptor, as can be seen in Table 2. The compounds shown in Table 2 were also tested at the adenosine A_{2A} and A₃ receptors and were shown to be generally selective for the adenosine A₁ receptor.

TABLE 2
Radioligand Binding Assay

| Comp | R^{″} | A₁^{a} |
|---|---|---|
| 4 | Me | 13.9 ± 5.4 |
| 6 | Pr | 2.45 ± 0.23 |
| 7 | iPr | 0.61 ± 0.04 |
| 11 | cPent | 0.62 ± 0.30 |
| 12 | cHex | 0.90 ± 0.21 |

^{a}Displacement of specific [³H]DPCPX binding in CHO cells expressing human adenosine A₁ receptors. Kᵢ (nM) ± SEM (n = 3).

### LIST OF REFERENCES

1) B. B. Fredholm, A. P. IJzerman, K. A. Jacobson, K.-N. Klotz, J. Linden, Pharmacol. Rev. 2001, 53, 527-552.
2) P. J. M. van Galen, P. Nissen, I. van Wijngaarden, A. P. IJzerman, W. Soudijn, J. Med. Chem. 1991, 34, 1202-1206.
3) R. Sarges, H. R. Howard, R. G. Browne, L. A. Lebel, P. A. Seymour, et al., J. Med. Chem. 1990, 33, 2240-2254.
4) P. G. Baraldi, B. Cacciari, G. Spalluto, M. J. Pineda de las Infantas y Villatoro, C. Zocchi et al., J. Med. Chem. 1996, 39, 1164-1171.
5) P. G. Baraldi, B. Cacciari, R. Romagnoli, G. Spalluto, G. S. Moro, et al., J. Med. Chem. 2000, 43, 4768-4780.
6) S. Hess, C. E. Müller, W. Frobenius, U. Reith, K.-N. Klotz, et al., J. Med. Chem. 2000, 43, 4636-4646.
7) C. Senanayake, L. Fredenburgh, R. Reamer, J. Liu, R. Larsen, T. Verhoeven, P. Reider, Tetrahedron Lett. 1994, 35, 5775-5778.
8) N. Latif, N. Mishriky, N. S. Girgis, Indian J. Chem. 1981, 20B, 147-149.
9) A. Attia, M. Michael, Pharmazie 1982, 37, 551-553.
10) O. Prakash, H. Batra, H. Kaur, P. K. Sharma, V. Sharma, S. P. Singh, R. M. Moriarty, Synthesis 2001, 4, 541-543.
11) L. C. W. Chang, R. F. Spanjersberg, J. von Frijtag Drabbe Künzel, T. Mulder-Krieger, G. van den Hout, M. W. Beukers, J. Brussee, A. P. IJzerman, J. Med. Chem. 2004, 47, 6529-6540.

1. A compound having the structure of general formula (I): 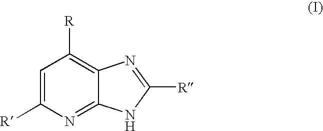
or a salt thereof,
wherein,
R represents hydrogen (except when R′=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl;
R′ represents hydrogen (except when R=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl;
R″ represents hydrogen, —(CH₂)ₙ-hydroxyl, halogen, acyl, thio-acyl, seleno-acyl, substituted alkyl, unsubstituted alkyl, substituted alkenyl, unsubstituted alkenyl, substituted alkynyl, unsubstituted alkynyl, substituted —(CH₂)ₙ-aryl, or unsubstituted —(CH₂)ₙ-aryl; and
n is a number in the range from 0 to 10.

2. The compound according to claim 1, wherein,
R represents hydrogen (except when R′=H), halogen, alkyl except —CH₃, substituted —(CH₂)ₙ-aryl, or unsubstituted —(CH₂)ₙ-aryl;
R′ represents hydrogen (except when R′=H), halogen, alkyl except —CH₃, or substituted —(CH₂)ₙ-aryl, or unsubstituted —(CH₂)ₙ-aryl;
R″ represents hydrogen, —(CH₂)ₙ-hydroxyl, halogen, acyl, thio-acyl, seleno-acyl, substituted alkyl, unsubstituted alkyl, substituted alkenyl, unsubstituted alkenyl, substituted alkynyl, unsubstituted alkynyl, substituted —(CH₂)ₙ-aryl, or unsubstituted —(CH₂)ₙ-aryl; and
n is a number in the range from 0 to 10.

3. The compound according to claim 1, wherein,
R represents a substituted —(CH₂)ₙ-aryl, or unsubstituted —(CH₂)ₙ-aryl;
R′ represents a substituted —(CH₂)ₙ-aryl, or unsubstituted —(CH₂)ₙ-aryl;
R″ represents hydrogen, —(CH₂)ₙ-hydroxyl, halogen, acyl, thio-acyl, seleno-acyl, substituted alkyl, unsubstituted alkyl, substituted alkenyl, unsubstituted alkenyl, substituted alkynyl, unsubstituted alkynyl, substituted —(CH₂)ₙ-aryl, or unsubstituted —(CH₂)ₙ-aryl; and
n is a number in the range from 0 to 10.

4. The compound according to claim 1, wherein,
R represents a substituted —(CH₂)ₙ-aryl, or unsubstituted —(CH₂)ₙ-aryl;
R′ represents a substituted —(CH₂)ₙ-aryl, or unsubstituted —(CH₂)ₙ-aryl;
R″ represents hydrogen, —(CH₂)ₙ-hydroxyl, halogen, substituted alkyl, unsubstituted alkyl, substituted —(CH₂)ₙ-aryl, or unsubstituted —(CH₂)ₙ-aryl; and
n is a number in the range from 0 to 10.

5. The compound according to claim 1, wherein,
R represents a substituted —(CH₂)ₙ-aryl, or unsubstituted —(CH₂)ₙ-aryl;
R′ represents a halogen;
R″ represents hydrogen, —(CH₂)ₙ-hydroxyl, halogen, substituted alkyl, unsubstituted alkyl, substituted —(CH₂)ₙ-aryl, or unsubstituted —(CH₂)ₙ-aryl; and
n is a number in the range from 0 to 10.

6. The compound according to claim 1, wherein
R represents a halogen;
R′ represents a substituted —(CH₂)ₙ-aryl, or unsubstituted —(CH₂)ₙ-aryl;
R″ represents hydrogen, —(CH₂)ₙ-hydroxyl, halogen, substituted alkyl, unsubstituted alkyl, substituted —(CH₂)ₙ-aryl, or unsubstituted —(CH₂)ₙ-aryl; and
n is a number in the range from 0 to 10.

7. The compound according to claim 1, wherein,
R represents a phenyl;
R′ represents a phenyl;
R″ represents —(CH₂)ₙ-hydroxyl, substituted —(CH₂)ₙ-aryl, unsubstituted —(CH₂)ₙ-aryl, hydrogen or an alkyl; and
n is a number in the range from 0 to 10.

8. The compound according to claim 1, wherein,
R represents a halogen;
R′ represents a phenyl;
R″ represents —(CH₂)ₙ-hydroxyl, halogen, substituted —(CH₂)ₙ-aryl, unsubstituted —(CH₂)ₙ-aryl, hydrogen or an alkyl; and
n is a number in the range from 0 to 10.

9. The compound according to claim 1, wherein,
R represents a phenyl;
R′ represents a halogen;
R″ represents —(CH₂)ₙ-hydroxyl, halogen, substituted —(CH₂)ₙ-aryl, unsubstituted —(CH₂)ₙ-aryl, hydrogen or an alkyl; and
n is a number in the range from 0 to 10.

10. The compound according to claim 1, wherein,
R represents a substituted alkyl except —CH₃, or unsubstituted alkyl except —CH₃;
R′ represents a substituted alkyl except —CH₃, or unsubstituted alkyl except —CH₃;
R″ represents hydrogen, halogen, —(CH₂)ₙ-hydroxyl, substituted alkyl, unsubstituted alkyl, substituted —(CH₂)ₙ-aryl, or unsubstituted —(CH₂)ₙ-aryl; and
n is a number in the range from 0 to 10.

11. The compound according to claim 1, wherein,
R represents halogen;
R′ represents a substituted alkyl except —CH₃, or unsubstituted alkyl except —CH₃;
R″ represents hydrogen, —(CH₂)ₙ-hydroxyl, halogen, substituted alkyl, or unsubstituted alkyl; and
n is a number in the range from 0 to 10.

12. The compound according to claim 1, wherein,
R represents a substituted alkyl except —CH₃, or unsubstituted alkyl except —CH₃;
R′ represents halogen;
R″ represents a hydrogen, —(CH₂)ₙ-hydroxyl, halogen, substituted alkyl, unsubstituted alkyl, substituted —(CH₂)ₙ-aryl, or unsubstituted —(CH₂)ₙ-aryl; and
n is a number in the range from 0 to 10.

13. The compound according to claim 1, wherein the compound is selected from the group consisting of 2,6-Diphenyl-1-deazapurine, 2,6-Bis(4-chlorophenyl)-1-deazapurine, 2,6-Bis(4-tolyl)-1-deazapurine, 2,6-Bis(4-methoxyphenyl)-1-deazapurine, 2-(4-chlorophenyl)-6-phenyl-1-deazapurine, 2-Tolyl-6-phenyl-1-deazapurine, 2-(4-methoxyphenyl)-6-phenyl-1-deazapurine, 6-Chloro-2,8-diphenyl-1-deazapurine, 2-Phenyl-6-chloro-8-propyl-1-deazapurine, 2-Phenyl-6-chloro-8-isopropyl-1-deazapurine, 2-Phenyl-6-chloro-8-cyclopentyl-1-deazapurine, 2-Phenyl-6-chloro-8-cyclohexyl-1-deazapurine, 2,6,8-Triphenyl-1-deazapurine, 8-Propyl-2,6-diphenyl-1-deazapurine, 8-Isopropyl-2,6-diphenyl-1-deazapurine, 8-Cyclopentyl-2,6-diphenyl-1-deazapurine, 8-Cyclohexyl-2,6-diphenyl-1-deazapurine, 8-Cyclohexyl-6-(4-chlorophenyl)-2-phenyl-1-deazapurine, 8-Cyclohexyl-6-(3,4-dichlorophenyl)-2-phenyl-1-deazapurine, 8-Cyclohexyl-6-(4-tolyl)-2-phenyl-1-deazapurine, 8-Cyclohexyl-6-(4-methoxyphenyl)-2-phenyl-1-deazapurine, 8-Furan-2-yl-2,6-diphenyl-1-deazapurine, 2,6-Diphenyl-8-thien-2-yl-1-deazapurine and salts thereof.

14. The compound according to claim 1, wherein the compound is selected from the group consisting of 2,6-Diphenyl-1-deazapurine, 2,6,8-Triphenyl-1-deazapurine, 8-Propyl-2,6-diphenyl-1-deazapurine, 8-Isopropyl-2,6-diphenyl-1-deazapurine, 8-Cyclopentyl-2,6-diphenyl-1-deazapurine, 8-Cyclohexyl-2,6-diphenyl-1-deazapurine, 8-Cyclohexyl-6-(4-chlorophenyl)-2-phenyl-1-deazapurine, 8-Cyclohexyl-6-(3,4-dichlorophenyl)-2-phenyl-1-deazapurine, 8-Cyclohexyl-6-(4-tolyl)-2-phenyl-1-deazapurine, 8-Cyclohexyl-6-(4-methoxyphenyl)-2-phenyl-1-deazapurine and salts thereof.

15. The compound according to claim 1, wherein the compound comprises 8-Cyclopentyl-2,6-diphenyl-1-deazapurine, 8-Isopropyl-2,6-diphenyl-1-deazapurine, 8-propyl-2,6-diphenyl-1-deazapurine, 8-methyl-2,6-diphenyl-1-deazapurine 8-Cyclohexyl-2,6-diphenyl-1-deazapurine, 8-Furan-2-yl-2,6-diphenyl-1-deazapurine, or 2,6-Diphenyl-8-thien-2-yl-1-deazapurine or a salt of any thereof.

16. A process for preparing a compound according to claim 1, which process comprises the steps of:
(a) i) reacting a compound having the structure R′COCH₂COR with malonamamidine, or a salt thereof, or, alternatively,
ii) reacting a compound having the structure R′COCH═CHR with malononitrile, or a salt thereof, yielding intermediate structure: 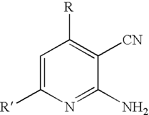
with subsequent hydrolysis of the cyano group, to finally form a product having the structure; 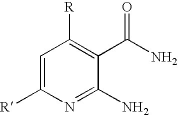
wherein,
R represents hydrogen (except when R′=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl; and
R′ represents hydrogen (except when R=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl; and n is a number in the range from 0 to 10;

16. A process for preparing a compound according to any one of claims 1-16, which process comprises the steps of:
(a) i) reacting a compound having the structure R′COCH₂COR with malonamamidine, or a salt thereof, or, alternatively,
ii) reacting a compound having the structure R′COCH═CHR with malononitrile, or a salt thereof, yielding intermediate structure: 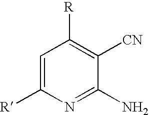
with subsequent hydrolysis of the cyano group, to finally form a product having the structure: 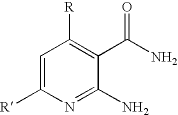
wherein,
R represents hydrogen (except when R′=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl; and
R′ represents hydrogen (except when R=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl; and n is a number in the range from 0 to 10;
(b) subjecting the final product formed in step (a) to a Hoffmann rearrangement reaction to form a product having the structure: 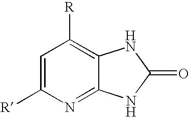
isomeric to structure: 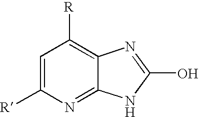
wherein
R, R′ and n have the meaning as defined in step (a);
(c) subjecting the product formed in step (b) to a mixture of an appropriate acid and its corresponding acid anhydride to form a product having the structure: 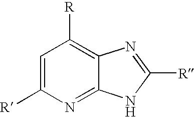
wherein,
R R′ and n have the meaning as defined in step (a); and
R represents hydrogen (except when R′=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl; and
R′ represents hydrogen (except when R=H), halogen, substituted alkyl except —CH₃, unsubstituted alkyl except —CH₃, substituted alkenyl, unsubstituted alkenyl, substituted alkynyl, unsubstituted alkynyl, substituted —(CH₂)ₙ-aryl, or unsubstituted —(CH₂)ₙ-aryl; and
R″ represents hydrogen, —(CH₂)ₙ-hydroxyl, halogen, acyl, thio-acyl, seleno-acyl, substituted alkyl, unsubstituted alkyl, substituted alkenyl, unsubstituted alkenyl, substituted alkynyl, unsubstituted alkynyl, substituted —(CH₂)ₙ-aryl, or unsubstituted —(CH₂)ₙ-aryl.

17. A method for treating and/or preventing a disorder in which the adenosine receptors are involved, the method comprising:
administering to a subject a composition according to claim 1.

18. A medicament comprising a compound according to claim 1.

19. A pharmaceutical composition comprising as an active ingredient a compound according to claim 1.

20. A method for treating and/or preventing a disorder in which the interaction with the adenosine receptors is beneficial, the method comprising:
administrating to a subject in need of such treatment an effective dose of a pharmaceutical composition according to claim 19.
